(19) United States
(12) Patent Application Publication
(10) Pub. No.: US 2026/0234326 A1
(43) Pub. Date: Aug. 13, 2026

(54) UV LIGHT STABILIZERS

(71) Applicant: BASF SE, Ludwigshafen am Rhein (DE)

(72) Inventors: Barbara WINKLER, Schweizerhalle (Muttenz) (CH); Wolfgang PETER, Ludwigshafen am Rhein (DE); Anna Maria CRISTADORO, Lemfoerde (DE)

(21) Appl. No.: 19/472,282

(22) PCT Filed: Mar. 28, 2024
(86) PCT No.: PCT/EP2024/058431

§ 371 Date: Oct. 3, 2025

(30) Foreign Application Priority Data
Apr. 5, 2023 (EP) 23166753.6

(51) Int. Cl. C08G 65/333

(52) U.S. Cl. CPC C08G 65/33396

(57) ABSTRACT

Method for producing polyetherols, comprising the following steps: a) reacting at least one starter compound with at least one alkylene oxide and a catalyst to yield a raw polyetherol, and b) removing low-molecular by-products from the raw polyetherol from step a), characterized in that the crude polyetherol is treated with a stripping gas in a column having internals.

### FIELD OF INVENTION

The presently claimed invention is directed to a ultraviolet (UV) absorbing compounds and process for the preparation thereof.

### BACKGROUND OF THE INVENTION

Ultraviolet (UV) light absorbers are an important class of organic compounds which find application in the protection and stabilization of organic materials such as plastics, polymers and coating materials against damages by light, heat, oxygen, or environmental forces. Such stabilizers are also widely used in personal care, fibers, paints, dyes among others.

These stabilizers protect materials by converting absorbed ultraviolet rays into low-impact heat and energy. The stabilizers are mainly used to curtail photodegradation (for instance in plastics or polymers) by playing a key role in suppressing the generation of radicals that cause plastic to degrade. While, both UVA and UVB are known to initiate material degradation, owing to the greater abundance of UVA in the sunlight, its contribution towards of such degradation is recognized to be greater. In this regard, the two commonly employed class of UV stabilizer applications that can provide appreciable protection against UVA and also UVB, are substituted benzotriazoles (BTZs) and substituted triazines, such as hydroxy phenyl triazines (HPTs).

The literature is abundant on the use of such triazine and benzotriazole-based stabilizers for ultraviolet stabilization. For instance, WO 2019006750 A1 describes the use of reactive benzotriazoles based on mono-trimethylolpropane, mainly for stabilization of polyurethanes against UV degradation. More recently, US2017/0349730 A1 introduces a composition comprising hydroxyphenyl triazines and hindered amine stabilizers in combination with antioxidants and phosphite into molded articles to obtain suitable thermal and ultraviolet stabilization.

However, several challenges have been identified in this field. For instance, the low molecular weight of well-known stabilizers makes them highly incompatible with use in plastics, leading to high level of migration of these molecules towards the boundaries of the material, thereby resulting in poor long-term stability of the material. This is countered in WO 2010130752 A1 by the synthesis of stabilizer moieties bearing multiple benzotriazole moieties. The compounds are also noted to display improved integration within the polymeric matrix, thereby resulting in improved stability.

However, despite the mentioned improvements, there are additional challenges associated with the field. For instance, the typical stabilizer compounds may suffer from poor solubility in common organic solvent, such as xylene. The absence of sufficient solubility renders the compound economically and environmentally unviable for industrial application. Additionally, amidst growing environmental concerns, the environmental impact associated with the synthesis of chemical compounds is a very relevant problem in today's age. In this regard, obtaining stabilizers that pose limited burden on the environment are highly sought after.

Therefore, there is an unmet need to develop UV stabilizer compounds that are environmentally benign, while maintaining important performance parameters such as high solubility, processability and stability.

### SUMMARY OF THE INVENTION

Surprisingly it was found that UV absorbers bound to polyether polyol obtained by the reaction of at least one sugar and polyhydric alcohol with alkylene oxide and optionally fatty acid can improve their solubility in organic solvents such as xylene, in addition to improving long-term stability. Additionally, the incorporation of biologically relevant sugars improves the environmental impact of the UV absorbers.

Accordingly, the first aspect of the presently claimed invention is directed to a product, or a salt thereof obtainable by a process comprising the step of:
a) reacting at least one compound of formula (I) in the presence of at least one catalyst,

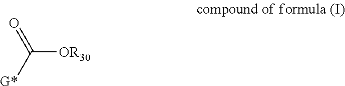

wherein G* is selected from a substituted 2-(2-hydroxyphenyl)-2H-benzotriazole group or substituted 2-hydroxyphenyl-s-triazine,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl;
b) with polyether polyol obtainable by reacting a composition of:
b1) 5 to 90 wt % based on the total amount of the polyether polyol, of at least one sugar selected from C₅-C₆ sugar alcohols, monosaccharides, oligosaccharides, polysaccharides, or mixtures thereof;
b2) 5 to 90 wt % based on the total amount of the polyether polyol, of at least one alkylene oxide;
b3) 0 to 90 wt % based on the total amount of the polyether polyol, of at least one polyhydric alcohols having 2 to 4 hydroxy groups; and
b4) 0 to 15 wt % based on the total amount of the polyether polyol, of at least one optional reactant selected from fatty acid, fatty acid monoesters or mixtures thereof.

The second aspect of the presently claimed invention is directed to process for obtaining the product or a salt thereof according first aspect as ultraviolet stabilizer

The third aspect of the presently claimed invention is directed to use of the product or a salt thereof according first aspect as ultraviolet stabilizer.

The fourth aspect of the presently claimed invention is directed to a composition comprising a product or a salt thereof according first aspect.

The fifth aspect of the presently claimed invention is directed to a method of protecting a material or coating from light, wherein the method comprises a step of providing the product or salt thereof as UV stabilizer according to first aspect.

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

In the context of the present invention, the product is useful as a ultraviolet (UV) stabilizer or light stabilizers. Said term, may be defined as products or compounds with ability to absorb light in the UVA and/or UVB. As mentioned above, the stabilizers are mainly used to curtail photodegradation (for instance in plastics or polymers) by playing a key role in suppressing the generation of radicals that cause plastic to degrade. Suitable stabilizers typically have at least one absorption maximum in the range between 280 to 420 nm.

Within the context of the presently claimed invention, the term “alkyl”, as used herein, refers to an acyclic saturated aliphatic group, including linear or branched alkyl saturated hydrocarbon radicals, denoted by a general formula CₙH₂ₙ₊₁ and wherein n is the number of carbon atoms such as 1, 2, 3, 4, etc.

Within the context of the presently claimed invention, the unsubstituted linear C₁-C₂₄ alkyl is preferably selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; more preferably selected from the group consisting of hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; even more preferably selected from the group consisting of methyl, ethyl, propyl, butyl, pentyl, hexyl, heptyl, octyl, nonyl, decyl, undecyl, dodecyl, tridecyl, tetradecyl and pentadecyl; most preferably selected from the group consisting of methyl, ethyl, propyl, butyl, pentyl, hexyl, heptyl, octyl, nonyl, decyl, undecyl and dodecyl; and in particular selected from the group consisting of methyl, ethyl, propyl, butyl, pentyl, hexyl, heptyl, octyl, nonyl and decyl.

Within the context of the presently claimed invention, the unsubstituted branched C₁-C₂₄ alkyl is preferably selected from the group consisting of isopropyl, iso-butyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl, more preferably selected from the group consisting of 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl, iso-eicosyl, 2-methyltricosyl, 2-ethyldocosyl, 3-ethylhenicosyl, 3-ethylicosyl, 4-propylhenicosyl, propylnonadecyl, 6-butyldodecyl and 5-ethylundecyl.

Within the context of the presently claimed invention, the substituted, linear or branched C₁-C₂₄ alkyl refers to a branched or linear saturated hydrocarbon group having C₁-C₂₄ carbon atoms substituted with functional groups selected from the group consisting of hydroxy, alkoxy, C(═O)—R, CN and SR, wherein R is selected from the group consisting of hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl and substituted or unsubstituted C₇-C₂₄ arylalkyl.

Within the context of the presently claimed invention, the substituted, linear or branched C₁-C₂₄ alkyl is preferably selected from the group consisting of 1-hydroxy methyl, 1-methoxy methyl, 1-hydroxy ethyl, 1-hydroxy propyl, 1-hydroxy butyl, 1-hydroxy pentyl, 1-hydroxy hexyl, 1-hydroxy heptyl, 1-hydroxy octyl, 1-hydroxy nonyl, decyl, 1-hydroxy undecyl, 1-hydroxy dodecyl, 1-hydroxy tridecyl, 1-hydroxy tetradecyl, 1-hydroxy pentadecyl, 1-hydroxy hexadecyl, 1-hydroxy heptadecyl, 1-hydroxy octadecyl, 1-hydroxy nonadecyl, 1-hydroxy eicosyl, 1-hydroxy henicosyl, 1-hydroxy docosyl, 1-hydroxy tricosyl, 1-hydroxy tetracosyl, 1-methoxy methyl, 1-methoxy ethyl, 1-methoxy propyl, 1-methoxy butyl, 1-methoxy pentyl, 1-methoxy hexyl, 1-methoxy heptyl, 1-methoxy octyl, 1-methoxy nonyl, decyl, 1-methoxy undecyl, 1-methoxy dodecyl, 1-methoxy tridecyl, 1-methoxy tetradecyl, 1-methoxy pentadecyl, 1-methoxy hexadecyl, 1-methoxy heptadecyl, 1-methoxy octadecyl, 1-methoxy nonadecyl, 1-methoxy eicosyl, 1-methoxy henicosyl, 1-methoxy docosyl, 1-methoxy tricosyl, 1-methoxy tetracosyl, 2-methoxy propyl, 2-methoxy butyl, 2-methoxy pentyl, 2-methoxy hexyl, 2-methoxy heptyl, 2-methoxy octyl, 2-methoxy nonyl, decyl, 2-methoxy undecyl, 2-methoxy dodecyl, 2-methoxy tridecyl, 2-methoxy tetradecyl, 2-methoxy pentadecyl, 2-methoxy hexadecyl, 2-methoxy heptadecyl, 2-methoxy octadecyl, 2-methoxy nonadecyl, 2-methoxy eicosyl, 2-methoxy henicosyl, 2-methoxy docosyl, 2-methoxy tricosyl, 2-methoxy tetracosyl, 1-acetoxy methyl, 1-acetoxy ethyl, 1-acetoxy propyl, 1-acetoxy butyl, 1-acetoxy pentyl, 1-acetoxy hexyl, 1-acetoxy heptyl, 1-acetoxy octyl, 1-acetoxy nonyl, decyl, 1-acetoxy undecyl, 1-acetoxy dodecyl, 1-acetoxy tridecyl, 1-acetoxy tetradecyl, 1-acetoxy pentadecyl, 1-acetoxy hexadecyl, 1-acetoxy heptadecyl, 1-acetoxy octadecyl, 1-acetoxy nonadecyl, 1-acetoxy eicosyl, 1-acetoxy henicosyl, 1-acetoxy docosyl, 1-acetoxy tricosyl, 1-acetoxy tetracosyl, 1-cyano methyl, 1-cyano ethyl, 1-cyano propyl, 1-cyano butyl, 1-cyano pentyl, 1-cyano hexyl, 1-cyano heptyl, 1-cyano octyl, 1-cyano nonyl, decyl, 1-cyano undecyl, 1-cyano dodecyl, 1-cyano tridecyl, 1-cyano tetradecyl, 1-cyano pentadecyl, 1-cyano hexadecyl, 1-cyano heptadecyl, 1-cyano octadecyl, 1-cyano nonadecyl, 1-cyano eicosyl, 1-cyano henicosyl, 1-cyano docosyl, 1-cyano tricosyl, 1-cyano tetracosyl, 2-cyano propyl, 2-cyano butyl, 2-cyano pentyl, 2-cyano hexyl, 2-cyano heptyl, 2-cyano octyl, 2-cyano nonyl, decyl, 2-cyano undecyl, 2-cyano dodecyl, 2-cyano tridecyl, 2-cyano tetradecyl, 2-cyano pentadecyl, 2-cyano hexadecyl, 2-cyano heptadecyl, 2-cyano octadecyl, 2-cyano nonadecyl, 2-cyano eicosyl, 2-cyano henicosyl, 2-cyano docosyl, 2-cyano tricosyl, 2-cyano tetracosyl, 1-thioyl methyl, 1-thioyl ethyl, 1-thioyl propyl, 1-thioyl butyl, 1-thioyl pentyl, 1-thioyl hexyl, 1-thioyl heptyl, 1-thioyl octyl, 1-thioyl nonyl, decyl, 1-thioyl undecyl, 1-thioyl dodecyl, 1-thioyl tridecyl, 1-thioyl tetradecyl, 1-thioyl pentadecyl, 1-thioyl hexadecyl, 1-thioyl heptadecyl, 1-thioyl octadecyl, 1-thioyl nonadecyl, 1-thioyl eicosyl, 1-thioyl henicosyl, 1-thioyl docosyl, 1-thioyl tricosyl and 1-thioyl tetracosyl.

Within the context of the presently claimed invention, the term alkenyl denotes unsubstituted, linear C₂-C₂₄ alkenyl which is preferably selected from the group consisting of 1-propenyl, 1-butenyl, 1-pentenyl, 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyl and 2-eicosenyl, more preferably selected from 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyl and 2-eicosenyl, 20-henicosenyl, 2-docosenyl, 6-tricosenyl and 2-tetracosenyl.

Within the context of the presently claimed invention, the unsubstituted branched C₂-C₂₄ alkenyl is selected from the group consisting of isopropenyl, iso-butenyl, neo-pentenyl, 2-ethyl-hexenyl, 2-propyl-heptenyl, 2-butyl-octenyl, 2-pentyl-nonenyl, 2-hexyl-decenyl, iso-hexenyl, iso-heptenyl, iso-octenyl, iso-nonenyl, iso-decenyl, iso-dodecenyl, iso-tetradecenyl, iso-hexadecenyl, iso-octadecenyl, iso-eicosenyl, 2-methyl tricosenyl, 2-ethyl docosenyl, 3-ethylhenicosenyl, 3-ethyl icosenyl, 4-propylhenicosenyl, 4-propylnonadecenyl, 6-butyldodecenyl, 5-ethylundedcenyl, 1,4-hexadienyl, 1,3-hexadienyl, 2,5-hexadienyl, 3,5-hexadienyl, 2,4-hexadienyl, 1,3,5-hexatrienyl, 1,3,6-heptatrienyl, 1,4,7-octatrienyl or 2-methyl-1,3,5hexatrienyl, 1,3,5,7-octatetraenyl, 1,3,5,8-nonatetraenyl, 1,4,7,10-undecatetraenyl, 2-ethyl-1,3,6,8-nonatetraenyl, 2-ethenyl-1,3,5,8-nonatetraenyl, 1,3,5,7,9-decapentaenyl, 1,4,6,8,10-undecapentaenyl and 1,4,6,9,11-dodecapentaenyl.

Within the context of the presently claimed invention, substituted linear or branched C₂-C₂₄ alkenyl refers to a branched or a linear unsaturated hydrocarbon group having C₂-C₂₄ carbon atoms substituted with functional groups selected from, hydroxy, alkoxy, C(═O)—R, CN and SR; wherein R is hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl.

Within the context of the presently claimed invention, substituted linear or branched C₂-C₂₄ alkenyl is preferably selected from the group consisting of 2-hydroxy propenyl, 3-hydroxy butenyl, 3-hydroxy pentenyl, 5-hydroxy hexenyl, 7-hydroxy heptenyl, 3-hydroxy octenyl, 5-hydroxy nonenyl, decyl, 11-hydroxy undecenyl, 9-hydroxy dodecenyl, 6-hydroxy tridecenyl, 4-hydroxy tetradecenyl, 6-hydroxy pentadecenyl, 3-hydroxy hexadecenyl, 2-hydroxy heptadecenyl, 7-hydroxy octadecenyl, 6-hydroxy nonadecenyl, 4-hydroxy eicosenyl, 2-hydroxy henicosenyl, 3-hydroxy docosenyl, 2-hydroxy tricosenyl, 23-hydroxy tetracosenyl, 1-methoxy ethenyl, 2-methoxy propenyl, 4-methoxy butenyl, 3-methoxy pentenyl, 5-methoxy hexenyl, 2-methoxy heptenyl, 5-methoxy octenyl, 3-methoxy nonenyl, 6-methoxy undecenyl, 1-methoxy dodec-2-enyl, 1-methoxy tridec-5-enyl, 3-methoxy tetradic-5-enyl, 3-methoxy pentade-12-encyl, 10-methoxy hexadec-15-enyl, 12-methoxy heptadic-16-enyl, 1-methoxy octadec-3-enyl, 1-methoxy nonadec-2-enyl, 1-methoxy eicos-20-enyl, 1-methoxy henicos-2-enyl, 1-methoxy docos-4-enyl, 1-methoxy tricos-22-enyl, 1-methoxy tetracos-23-enyl, 2-methoxy prop-1-enyl, 2-methoxy but-1-enyl, 2-methoxy pent-4-enyl, 2-methoxy hex-2-enyl, 2-methoxy hept-3-enyl, 2-methoxy oct-7-enyl, 2-methoxy non-5-enyl, 2-methoxy undec-10-enyl, 2-methoxy dodec-4-enyl, 2-methoxy tridec-12-enyl, 2-methoxy tetradic-10-enyl, 2-methoxy pentadec-14-enyl, 2-methoxy hexadec-1-enyl, 2-methoxy heptadic-1-enyl, 2-methoxy octadic-12-enyl, 2-methoxy nonadec-10-enyl, 2-methoxy eicos-18-enyl, 2-methoxy henicos-2-enyl, 2-methoxy docos-3-enyl, 20-methoxy tricos-2-enyl, 21-methoxy tetracos-4-enyl, 1-acetoxy ethenyl, 1-acetoxy prop-1-enyl, 1-acetoxy but-2-enyl, 1-acetoxy pent-4-enyl, 1-acetoxy hex-2-enyl, 1-acetoxy hept-1-enyl, 1-acetoxy oct-7-enyl, 1-acetoxy non-2-enyl, 5-acetoxy dec-3-enyl, 1-acetoxy undec-10-enyl, 1-acetoxy dodec-2-enyl, 1-acetoxy tridec-12-enyl, 10-acetoxy tetradec-2-enyl, 15-acetoxy pentadec-2-enyl, 10-acetoxy hexadec-2-enyl, 11-acetoxy heptadec-1-enyl, 13-acetoxy octadec-2-enyl, 1-acetoxy nonadec-14-enyl, 20-acetoxy eicos-19-enyl, 1-acetoxy henicos-2-enyl, 1-acetoxy docos-10-enyl, 1-acetoxy tricos-22-enyl, 1-acetoxy tetracos-23-enyl, 1-cyano eth-1-enyl, 1-cyano prop-2-enyl, 1-cyano but-2-enyl, 1-cyano pent-3-enyl, 1-cyano hex-5-enyl, 1-cyano hept-6-enyl, 1-cyano oct-2-enyl, 1-cyano non-3-enyl, 11-cyano undec-2-enyl, 10-cyano dodec-2-enyl, 10-cyano tridec-12-enyl, 1-cyano tetradec-3-enyl, 1-cyano pentadec-14-enyl, 1-cyano hexadec-15-enyl, 1-cyano heptadec-2-enyl, 1-cyano octadec-3-enyl, 1-cyano nonadec-18-enyl, 1-cyano eicos-10-enyl, 1-cyano henicos-20-enyl, 15-cyano docos-3-enyl, 1-cyano tricos-20-enyl, 1-cyano tetracos-2-enyl, 2-cyano prop-2-enyl, 2-cyano but-1-enyl, 2-cyano pent-1-enyl, 2-cyano hex-3-enyl, 2-cyano hept-6-enyl, 2-cyano oct-1-enyl, 2-cyano non-8-enyl, 2-cyano undec-10-enyl, 2-cyano dodec-1-enyl, 2-cyano tridec-12-enyl, 2-cyano tetradec-10-enyl, 2-cyano pentadec-3-enyl, 2-cyano hexadec-2-enyl, 2-cyano heptadec-1-enyl, 2-cyano octadec-12-enyl, 2-cyano nonadec-15-enyl, 2-cyano eicos-1-enyl, 2-cyano henicos-5-enyl, 2-cyano docos-20-enyl, 2-cyano tricos-22-enyl, 2-cyano tetracos-20-enyl, 1-thionyl eth-1-enyl, 1-thionyl prop-2-enyl, 1-thionyl but-2-enyl, 1-thionyl pent-4-enyl, 1-thionyl hex-2-enyl, 1-thionyl hept-5-enyl, 1-thionyl oct-3-enyl, 1-thionyl non-5-enyl, 1-thionyl undec-10-enyl, 1-thionyl dodec-11-enyl, 1-thionyl tridec-2-enyl, 1-thionyl tetradec-4-enyl, 1-thionyl pentadec-5-enyl, 1-thionyl hexadec-3-enyl, 1-thionyl heptadec-2-enyl, 1-thionyl octadec-3-enyl, 1-thionyl nonadec-15-enyl, 1-thionyl eicos-18-enyl, 1-thionyl henicos-20-enyl, 1-thionyl docos-21-enyl, 1-thionyl tricos-20-enyl and 1-thionyl tetracos-22-enyl.

Within the context of the presently claimed invention, the substituted or unsubstituted C₅-C₂₄ cycloalkyl refers to a monocyclic and bicyclic 5- to 24-membered saturated cycloaliphatic radical. Representative examples of unsubstituted or branched C₅-C₂₄ monocyclic and bicyclic cycloalkyl include, but are not limited to, cyclopentyl, cyclohexyl, cycloheptyl, cyclooctyl, bicyclo[2.2.1]heptyl, and bicyclo[3.1.1]heptyl.

Within the context of the presently claimed invention, the C₅-C₂₄ monocyclic and bicyclic cycloalkyl can be further branched with one or more equal or different alkyl groups such as methyl, ethyl, n-propyl, iso-propyl, n-butyl, iso-butyl, n-pentyl, iso-pentyl, neo-pentyl etc. The representative examples of branched C₃-C₁₀ monocyclic and bicyclic cycloalkyl include, but are not limited to, methyl cyclohexyl and dimethyl cyclohexyl.

Within the context of the presently claimed invention, the unsubstituted or substituted C₅-C₂₄ cycloalkenyl refers to a monocyclic and bicyclic 5- to 24-membered unsaturated cycloaliphatic radical which comprises one or more double bonds. Representative examples of C₅-C₂₄ cycloalkenyl include, but are not limited to, cyclopentenyl, cyclohexenyl, cycloheptenyl, cyclooctenyl, cyclononenyl or cyclodecenyl. These radicals can be branched with one or more equal or different alkyl radical, preferably with methyl, ethyl, n-propyl or iso-propyl. The representative examples of branched C₅-C₂₄ monocyclic and bicyclic cycloalkenyl include, but are not limited to, methyl cyclohexenyl and dimethyl cyclohexenyl.

Within the context of the presently claimed invention, the substituted or unsubstituted C₆-C₂₄ aryl may have more than one aromatic ring. The representative examples for substituted and unsubstituted C₆-C₂₄ aryl include phenyl, naphthyl, anthracenyl, tetraphenyl, phenalenyl and phenanthrenyl.

Within the context of the presently claimed invention, the arylalkyl refers to an aryl ring attached to an alkyl chain. The representative examples for the arylalkyl include, but are not limited to, 1-phenylmethyl, 1-phenylethyl, 1-phenylpropyl, 1-phenylisopropyl, 1-phenylbutyl, 1-methyl-1-phenyl-propyl, 3-phenylpropyl, 4-phenylbutyl, 3-phenylbutyl and 2-methyl-3-phenyl-propyl.

Within the context of the presently claimed invention, the substituted C₆-C₂₄ aryl refers to an aromatic ring having substitution at different positions. The C₆-C₂₄ aryl may have more than one aromatic ring. The representative examples for substituted and unsubstituted C₆-C₂₄ aryl include tolyl, xylyl, 2-hydroxyphenyl, 2,3-dihydroxyphenyl, 2-methoxy phenyl, 2-hydroxy-4-methoxyphenyl, 2,4-dimethoxyphenyl, 2-chlorophenyl, 2-chloro-4-hydroxyphenyl, 2-chloro-4-methoxy phenyl, 3-chloro-4-methoxyphenyl, 2-methyl-4-methoxy-6-chlorophenyl and 2-acetyl-4-hydroxyphenyl.

Within the context of the presently claimed invention, the term alkylene refers to an acyclic saturated hydrocarbon chains, which combine different moieties. The alkylene refers to linear unsubstituted C₁ to C₃₀ carbon atoms, selected from, but not limited to, —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—, —CH₂—CH₂—CH₂—CH₂—CH₂—CH₂—CH₂—, —CH₂—CH₂—CH₂—CH₂—CH₂—CH₂—CH₂—CH₂—, —CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—.

Within the context of the present invention, the branched unsubstituted alkylene is selected from, but not limited to, —CH₂—C(CH₃)H—, —CH₂—C(CH₃)H—CH₂—, —CH₂—CH₂—C(CH₃)H—CH₂—, —C(CH₃)₂—CH₂—CH₂—C(CH₃)H—CH₂—, —CH₂—C(CH₃)H—CH₂—CH₂—CH₂—CH₂—, —CH₂—C(CH₃)H—CH₂—CH₂—CH₂—CH₂—CH₂—, —CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—, —CH₂—C(CH₃)₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—CH₂—CH₂—CH₂—, —CH₂—CH₂—CH₂—C(CH₃)H—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—.

Accordingly, the presently claimed invention is directed to a product, or a salt thereof obtainable by a process comprising the step of:
a) reacting at least one compound of formula (I) in the presence of at least one catalyst,

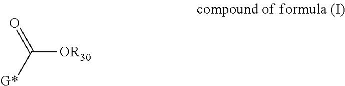

wherein G* is selected from a substituted 2-(2-hydroxyphenyl)-2H-benzotriazole group or substituted 2-hydroxyphenyl-s-triazine,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, substituted or unsubstituted, linear or branched C₁-C₂₄ heteroalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl;
b) with polyether polyol obtainable by reacting a composition of:
b1) 5 to 90 wt % based on the total amount of the polyether polyol, of at least one sugar selected from C₅—C sugar alcohols, monosaccharides, oligosaccharides, polysaccharides, or mixtures thereof;
b2) 5 to 90 wt % based on the total amount of the polyether polyol, of at least one alkylene oxide;
b3) 0 to 90 wt % based on the total amount of the polyether polyol, of at least one polyhydric alcohols having 2 to 4 hydroxy groups; and
b4) 0 to 15 wt % based on the total amount of the polyether polyol, of at least one optional reactant selected from fatty acid, fatty acid monoesters or mixtures thereof.

Preferably, R₃₀ is selected from hydrogen, substituted or unsubstituted, linear or branched C₁-C₂₄ alkyl, substituted or unsubstituted, linear or branched C₁-C₂₄ heteroalkyl, or substituted or unsubstituted C₇-C₁₆ arylalkyl.

More preferably, R₃₀ is selected from hydrogen, substituted or unsubstituted, linear or branched C₁-C₁₆ alkyl, substituted or unsubstituted, linear or branched C₁-C₁₆ heteroalkyl, or unsubstituted C₇-C₁₂ arylalkyl.

Even more preferably, R₃₀ is selected from hydrogen, unsubstituted, linear C₁-C₁₀ alkyl, or unsubstituted C₇-C₁₀ arylalkyl.

Preferably, G* is selected from substituted 2-hydroxyphenyl-s-triazine.

More preferably, the substituted 2-hydroxyphenyl-s-triazine is selected from formula (A),

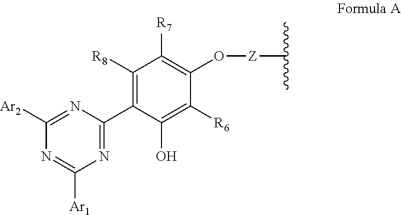

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

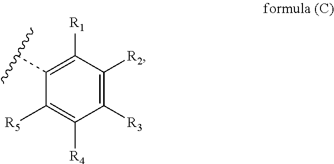

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, substituted or unsubstituted —OC₁-C₂₄ alkyl; or a moiety of formula (M),

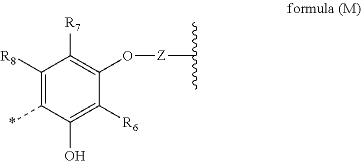

wherein the dotted line is a single bond between formula (M) and the triazinyl ring of formula (A), and Z is attached to —C(═O)OR₃₀,
R₆, R₇ and R₃ are independently of each other selected from hydrogen, or substituted or unsubstituted, linear or branched C₁-C₂₄ alkyl.

More preferably, at least one of Ar₁ and Ar₂ are independently of each other a moiety of the formula (C).

More preferably, R₁, R₂, R₃, R₄ and R₅ are independently of each other selected from hydrogen, OH, substituted or unsubstituted C₁-C₂₄ alkyl, substituted or unsubstituted C₆-C₂₄ aryl, or substituted or unsubstituted —O—C₁-C₂₄ alkyl.

More preferably, at least one of Ar₁ and Ar₂ in formula (A) are independently of each other a moiety of the formula (C-1)

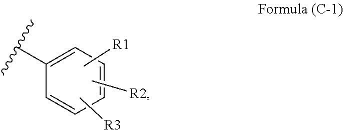

wherein the dotted line is a single bond between formula (C) and the triazinyl ring of formula (A), and R₁, R₂, and R₃ are independently of each other selected from hydrogen, substituted or unsubstituted, linear or branched C₁-C₂₄ alkyl, substituted or unsubstituted C₆-C₂₄ aryl, —OH, substituted or unsubstituted —OC₁-C₂₄ alkyl.

More preferably, at least one of Ar₁ and Ar₂ in formula (A) are independently of each other a moiety of the formula (C-1), wherein R₁, R₂, and R₃ are independently of each other selected from hydrogen, C₁-C₃ alkyl, unsubstituted C₆-C₁₂ aryl, —OH, or substituted or unsubstituted —O—C₁-C₁₆ alkyl.

More preferably, at least one of Ar₁ and Ar₂ in formula (A) are independently of each other a moiety of the formula (M).

More preferably, at least one of Ar₁ and Ar₂ in formula (A) are independently of each other a moiety of the formula (M), wherein R₆, R₇ and R₃ are independently of each other selected from hydrogen, or substituted or unsubstituted, linear or branched C₁-C₁₀ alkyl.

Even more preferably, at least one of Ar₁ and Ar₂ in formula (A) are independently of each other a moiety of the formula (M), wherein R₆, R₇ and R₃ are from hydrogen, or unsubstituted linear C₁-C₆ alkyl, more preferably from hydrogen or methyl.

Most preferably, the substituted 2-hydroxyphenyl-s-triazine is selected from formula (A) as described herein, wherein Z is selected from substituted or unsubstituted, linear or branched C₁-C₈ alkylene; R₁, R₂, R₃, R₄ and R₅ are independently of each other selected from hydrogen, unsubstituted C₁-C₆ alkyl, unsubstituted C₆-C₁₂ aryl, —OH, or —OC₁-C₆ alkyl; and R₆, R₇ and R₈ are hydrogen.

Preferably, G* is selected from a substituted 2-(2-hydroxyphenyl)-2H-benzotriazole group.

Preferably, substituted 2-(2-hydroxyphenyl)-2H-benzotriazole is selected from formula (B), Formula B

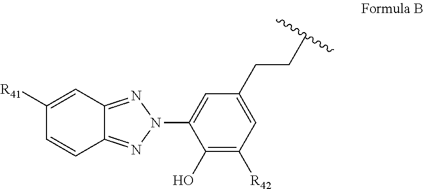

wherein R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

More preferably, substituted 2-(2-hydroxyphenyl)-2H-benzotriazole is selected from formula (B), wherein R₄₁, and R₄₂ independently of each other, are selected from hydrogen, halogen, substituted or unsubstituted, linear or branched C₁-C₁₀ alkyl or substituted or unsubstituted C₇-C₁₆ arylalkyl.

Even more preferably, substituted 2-(2-hydroxyphenyl)-2H-benzotriazole is selected from formula (B), wherein R₄₁, and R₄₂ independently of each other, are selected from hydrogen, halogen, unsubstituted, linear or branched C₁-C₆ alkyl or substituted or unsubstituted C₇-C₁₀ arylalkyl.

More preferably, G* is selected from compound of formula (A) of formula (B),

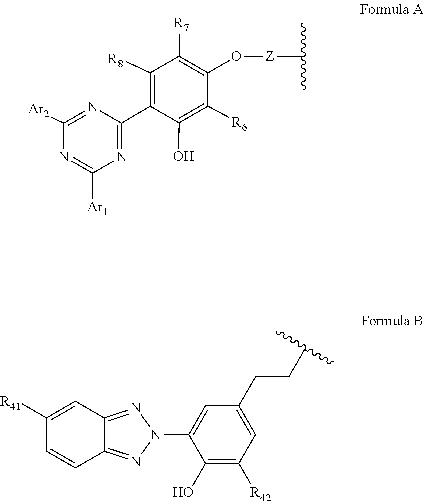

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

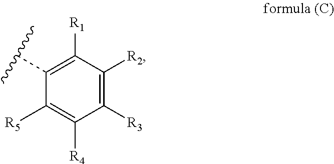

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, substituted or unsubstituted —OC₁-C₂₄ alkyl; or a moiety of formula (M),

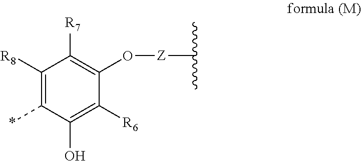

wherein the dotted line is a single bond between formula (M) and the triazinyl ring of formula (A), and Z is attached to —C(═O)OR₃₀,
R₆, R₇ and R₃ are independently of each other selected from hydrogen, or substituted or unsubstituted, linear or branched C₁-C₂₄ alkyl,
wherein R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

Halogen may be selected from fluoro, chloro, bromo, or iodo, preferably chloro.

More preferably, the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxyphenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methylphenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxyphenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, more preferably the compound of formula (I) according to presently claimed invention is selected from methyl 3-[3-(benzotriazol-2-yl)-5-tertbutyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tertbutyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxyphenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, most preferably the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, or a combination of two or more thereof, and in particular, according to presently claimed invention the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

More preferably, the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxyphenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, octyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate, 6-methylheptyl 2-[4-[4,6-bis(4-phenylphenyl)-1,3,5-triazin-2-yl]-3-hydroxyphenoxy]propanoate, octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, methyl 2-[4-[4,6-bis(2,4-dimethylphenyl)-1,3,5-triazin-2-yl]-2-methyl-3-hydroxy-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxyphenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof.

Even more preferably, the compound of formula (I) is selected from methyl 2-[4-[4,6-bis(2,4-dimethylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, octyl 2-[4-(4,6-diphenyl-1,3,5-triazin-2-yl)-3-hydroxy-phenoxy]propanoate, methyl 2-[4-[4,6-bis(4-phenylphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate, 6-methylheptyl 2-[4-[4,6-bis(4-phenylphenyl)-1,3,5-triazin-2-yl]-3-hydroxy-phenoxy]propanoate, methyl 2-[4-[4,6-bis(2,4-dimethylphenyl)-1,3,5-triazin-2-yl]-2-methyl-3-hydroxy-phenoxy]propanoate, methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

Most preferably, the compound of formula (I) is selected from

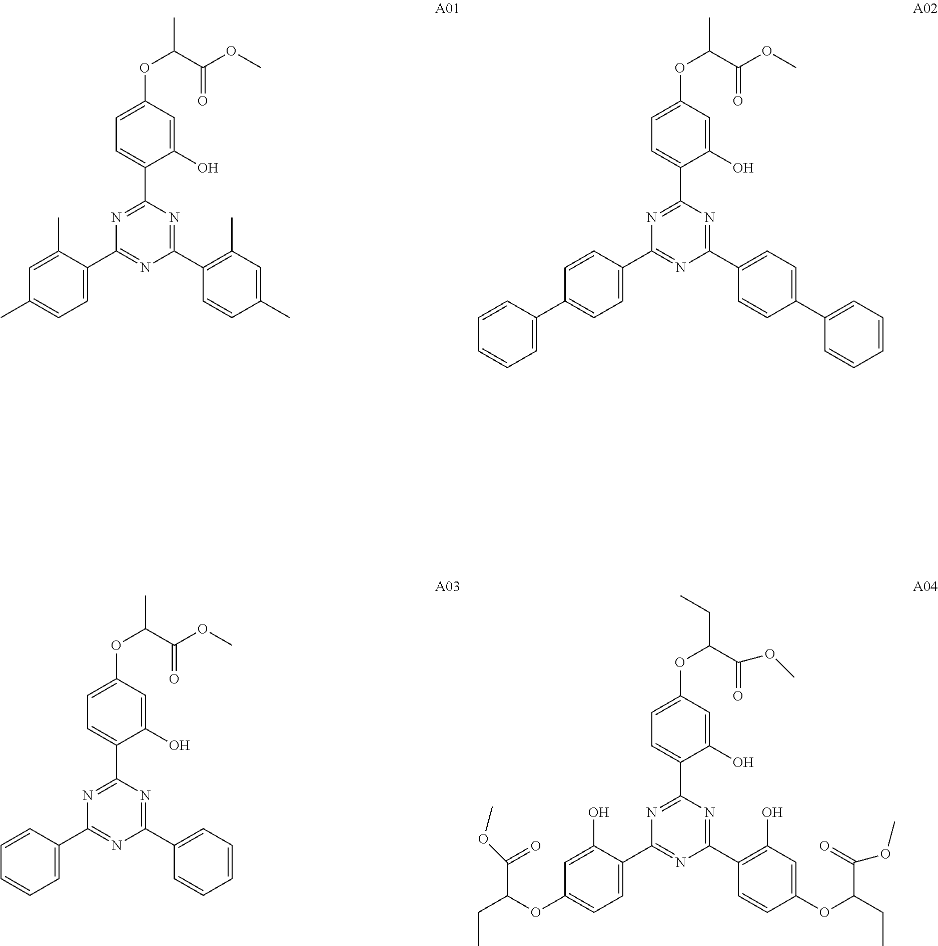

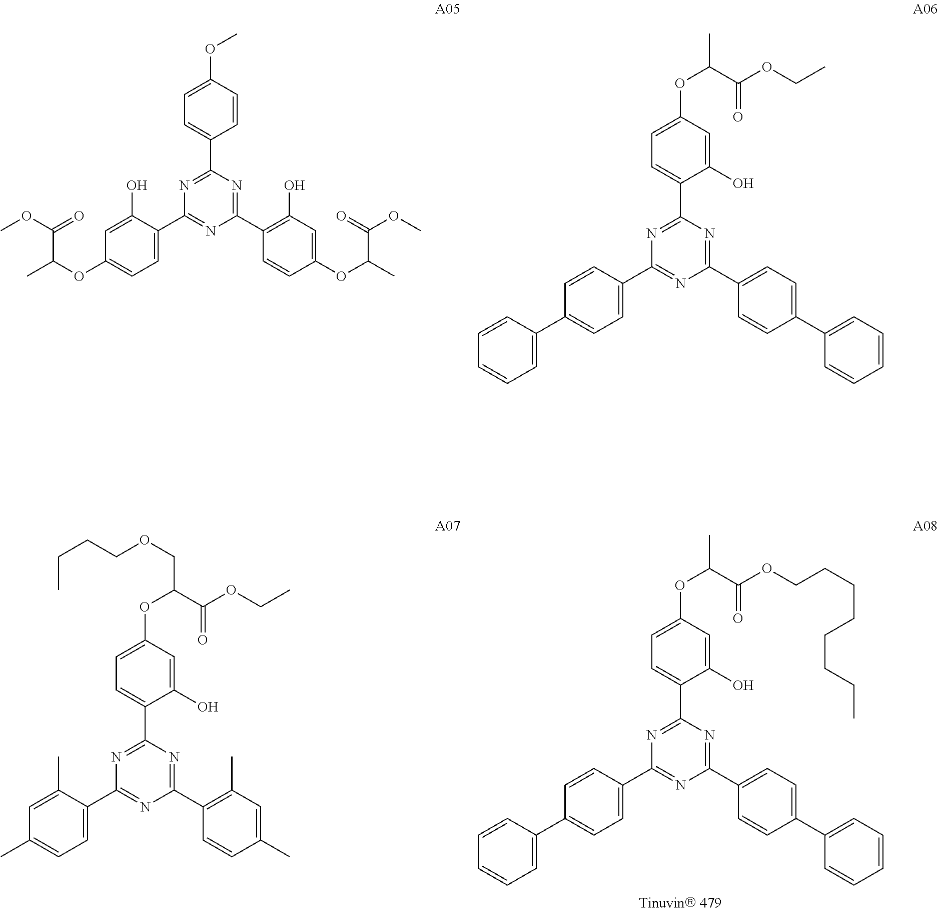

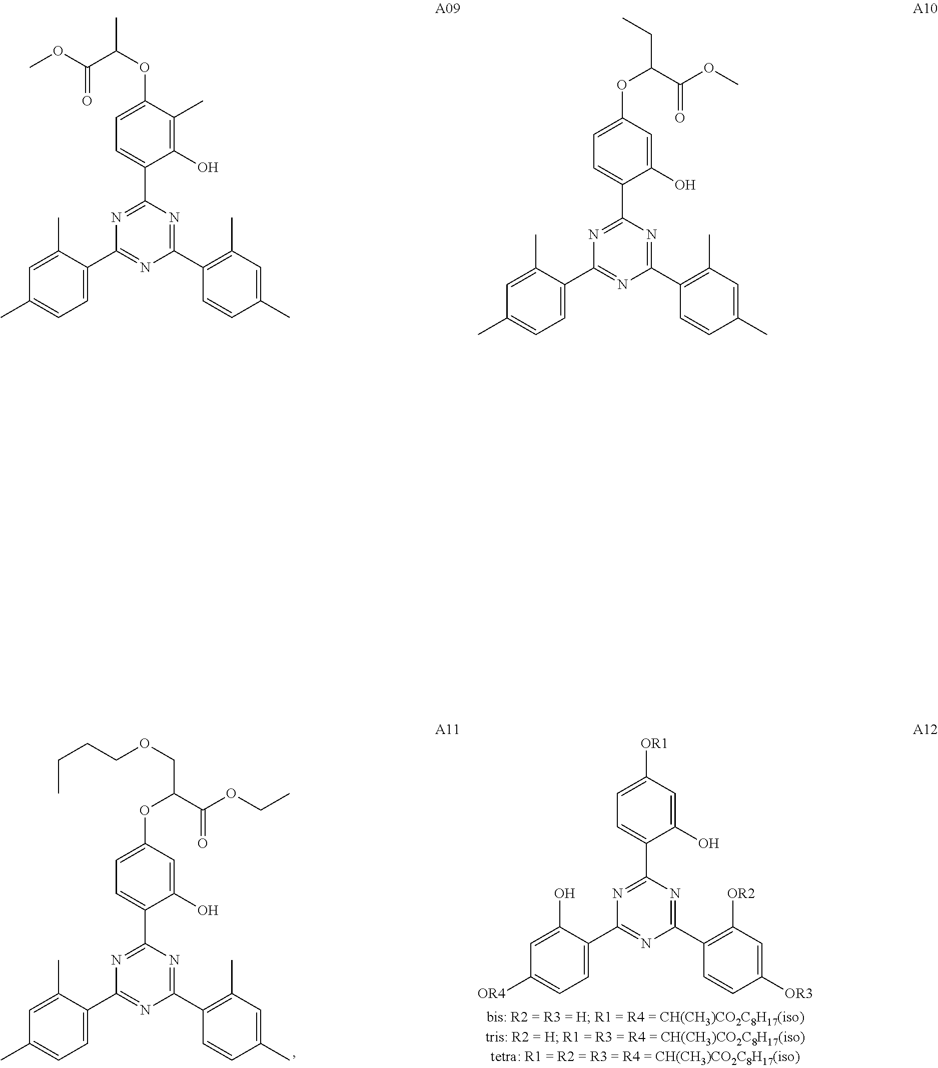

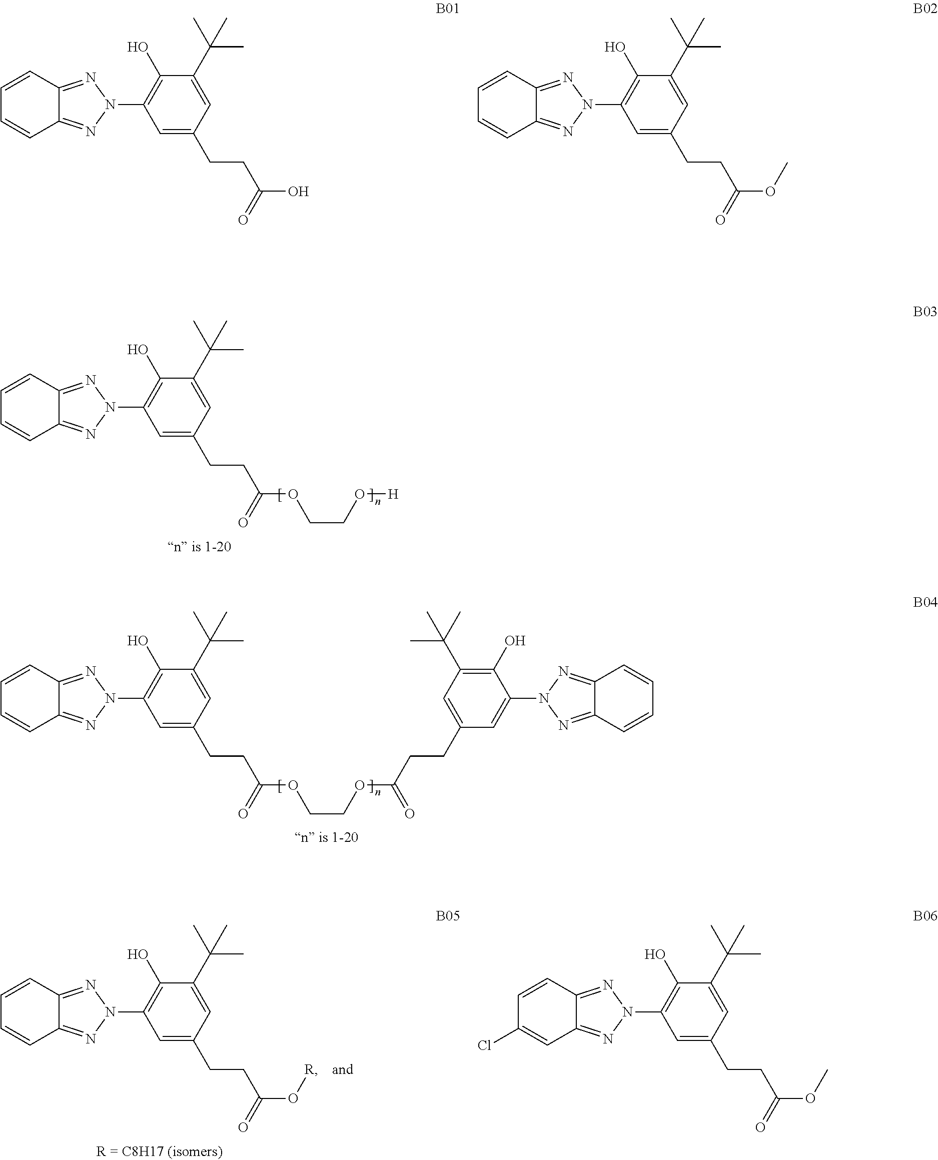

Preferably, polyether polyol obtainable by reacting a composition of:
b1) 5 to 80 wt %, more preferably 5 to 70 wt %, more preferably 10 to 60 wt % based on the total amount of the polyether polyol, of at least one sugar selected from C₅-C₆ sugar alcohols, monosaccharides, oligosaccharides, polysaccharides, or mixtures thereof;
b2) 5 to 80 wt %, more preferably 5 to 70 wt %, more preferably 10 to 60 wt % based on the total amount of the polyether polyol, of at least one alkylene oxide;
b3) 0 to 80 wt %, more preferably 0 to 70 wt %, more preferably 5 to 60 wt % based on the total amount of the polyether polyol, of at least one polyhydric alcohols having 2 to 4 hydroxy groups; and
b4) 0 to 12 wt %, more preferably 1 to 12 wt %, more preferably 2 to 10 wt % based on the total amount of the polyether polyol, of at least one optional reactant selected from fatty acid, fatty acid monoesters or mixtures thereof.

Preferably, the polyether polyol is obtained by a process in the presence of a catalyst selected from the group consisting of the aminic catalysts, or oxides, hydroxides or alkoxides of an alkali or alkaline earth metal. Said catalysts may be as defined in WO 2011107366 A1. The aminic catalysts are preferably selected from the group comprising trialkylamines, such as for example trimethylamine, triethylamine, tripropylamine and tributylamine; dimethylalkylamines, such as for example dimethylethanolamine, dimethylcyclohexylamine, dimethylethylamine and dimethylbutylamine; aromatic amines, such as for example dimethylaniline, dimethylaminopyridine, dimethylbenzylamine, pyridine, imdazoles, such as for example imidazole, 4(5)-methylimidazole, 3-methylimidazole and 1-hydroxypropylimidazole; guanidines and amidines, such as for example 1,5-diazabicyclo[4.3.0]non-5-ene and 1,5-diazabicyclo[5.4.0]undec-7-ene. The aminic catalyst is preferably selected from dimethylethanolamine and imidazole. The aminic catalyst is preferably used in an amount of 0.1-1.0 wt. % based on the total amount. This amount is particularly preferred when using aliphatic amines.

More preferably, the catalyst is selected from oxides, hydroxides or alkoxides of an alkali or alkaline earth metal. Said catalyst may be as described in WO2011107367 A1. Particularly preferably catalyst is selected from sodium hydroxide, potassium hydroxide, cesium hydroxide or potassium tert-butoxide Sugar, in the context of the present invention, may be defined as a chemical moiety bearing at least one monosaccharide unit with the general formula (CH₂O)ₓ, where typically 7≥x≥3. Preferred monosaccharides are 5 carbon (pentose) or 6 carbon (hexose) sugars. As is well known, such monosaccharides can chemically bond to one another via glycosidic linkages to yield short or long chain, (disaccharides, oligosaccharides or polysaccharides). Both monosaccharide as well as the bonded chain of saccharides (disaccharides, oligosaccharides or polysaccharides) are considered relevant for the present invention. Suitable polysaccharides may be as defined in WO2006040335 A1 and WO2006040333 A1.

A further classification of sugars may be on basis of availability of free aldose or ketose group that allows participation in reduction reactions. Reducing sugars such as glucose as well as non reducing sugars such as sucrose are considered part of the presently claimed invention.

In the context of the present invention, the sugar is selected from C₅-C₆ sugar alcohols, monosaccharides, oligosaccharides, polysaccharides, or mixtures thereof. C₅-C₆ sugar alcohols have at least 5 hydroxy groups, preferably 5-6 hydroxy groups.

Preferably, at least one sugar is selected from sucrose, sorbitol, xylitol, mannitol, galactitol or mixtures thereof, preferably from sucrose, sorbitol, or mixtures thereof.

The polyhydric alcohol is different from the sugar b1). Suitable polyhydric alcohols may be as described in WO2011012599 A1. More preferably, the polyhydric alcohol is selected from glycerol, monoethylene glycol, dietheylene glycol, triethylene glycol, 1,2-propanediol, 1,3-propanediol, dipropylene glycol, 1,4-butanediol, 1,6-hexanediol, pentaerythritol, trimethylolpropane, or mixtures thereof, preferably from glycerol, 1,2-propanediol, diethylene glycol, erythritol, pentaerythritol, trimethylolpropane, or mixtures thereof.

Suitable polyether polyol may be prepared by the oxyalkylation of at least one sugar. In principle, all suitable alkylene oxides can be used for the process according to the invention. Preferably, C₂-C₂₀ alkylene oxides, such as, for example, ethylene oxide, propylene oxide, 1,2-butylene oxide, 2,3-butylene oxide, isobutylene oxide, pentene oxide, hexene oxide, cyclohexene oxide, styrene oxide, dodecene epoxide, octadecene epoxide, and mixtures of these epoxides are suitable. Ethylene oxide, propylene oxide, 1,2-butylene oxide, 2,3-butylene oxide, isobutylene oxide and pentene oxide are more preferred, propylene oxide and ethylene oxide being even more preferred.

Suitable polyether polyols may be prepared by carrying out the described process in the presence of fatty acid, fatty acid monoesters or mixtures thereof. In the context of the present invention, the term “fatty acid, fatty acid monoesters or mixtures thereof” describes fatty acid glycerides, in particular fatty acid triglycerides, and/or fatty acid esters based on other mono- and polyfunctional alcohols. Suitable fatty acids or monoesters may be as listed in U.S. Pat. No. 9,284,401 B2 and US 2014/0200327 A1. The fatty acid radicals of the fatty acid esters can in their turn, as in the case of castor oil, carry hydroxyl groups. It is of course also possible to employ according to the invention fatty acid esters, the fatty acid radicals of which have been modified subsequently with hydroxyl groups. Fatty acid radicals modified in this way can be obtained, for example, by epoxidation of the olefinic double bonds and subsequent ring-opening of the oxirane rings by means of nucleophiles or by hydroformylation/hydrogenation. Unsaturated oils are often also treated with atmospheric oxygen at elevated temperature for this purpose.

In the context of the present invention, triglycerides are suitable and included under fatty acid, fatty acid monoesters or mixtures thereof. Particular examples may be cottonseed oil, groundnut oil, coconut oil, linseed oil, palm kernel oil, olive oil, maize-oil, palm oil, castor oil, lesquerella oil, rapeseed oil, soya oil, sunflower oil, herring oil, sardine oil, tallow and lard. Fatty acid esters of other mono- or polyfunctional alcohols and fatty acid glycerides having less than 3 fatty acid radicals per glycerol molecule can of course also be employed according to the invention. The fatty acid (tri)glycerides and the fatty acid esters of other mono- and polyfunctional alcohols can also be employed in the mixture.

Preferably, suitable polyether polyols may be prepared by optional reaction in the presence of at least one compound selected from lauric acid, myristic acid, palmitic acid, stearic acid, palmitoleic acid, oleic acid, erucic acid, linoleic acid, underlie acid, elaeostearic acid, arachidonic acid and monoester thereof, preferably from stearic acid, oleic acid, or the methyl esters of the mentioned fatty acids.

The reaction conditions and parameters required to obtain said polyether polyols are well-known. These may for instance be found in PCT/EP2022/055942, EP 2542612 among others.

Preferably, the polyether polyol has average number of hydroxy groups in the range of 1 to 25, more preferably in the range of 1 to 20, even more preferably in the range of 1 to 15, most preferably in the range of 1 to 10, and in particular preferably in the range of 2 to 10.

During, the process for obtaining the polyether polyol, the components may be added simultaneously or in a stepwise manner. Preferably, the polyether polyol is obtained by a process wherein one or more of the b1), b2), b3) or b4) are added in a stepwise manner. More preferably, b1), b2), b3) are added simultaneously followed by b4).

Preferably, the polyether polyol has an OH number or hydroxyl number in the range from 50 to 2000 mg KOH/g, more preferably from 100 to 1500 mg KOH/g, most preferably from 200 to 800 mg KOH/g, measured according to DIN 53 240 (1971-12).

Preferably, the polyether polyol has a viscosity in the range from 1000 to 50000 mPa·S, more preferably from 1000 to 45000 mPa·S, even more preferably from 1000 to 30000 mPa·S, more preferably from 2000 to 30000 mPa·S, measured at 25° C. according to DIN EN 12092.

Preferably, the polyether polyol has a weight average molecular weight from 100 to 2500 g/mol, more preferably from 150 to 1000 g/mol, estimated with determined by gel permeation chromatography (GPC) using polystyrene as internal standard.

Preferably, the weight ratio of compound of formula (I) to the polyether polyol is in the range of 1 to 10 to 10 to 1, more preferably in the range of 1 to 7 to 7 to 1, most preferably in the range of 1 to 5 to 5 to 1.

The product, or a salt thereof of the present invention is obtained by a process as described herein, carried out in the presence of at least one catalyst.

Preferably, the catalyst is selected from protonic acids, Sn compound, Zr compound, Bi compound, Zn compound, Al compound, Ti compound, or a combination of two or more thereof.

Preferably, the Zr compound selected from zirconium acetate, zirconium octoate, zirconium 2-ethylhexanoate, zirconium decanoate, zirconium neodecanoate, bis(acetato-o)oxozirconium, bis(cyclopentadienyl)zirconium bis(trifluoromethanesulfonate) tetrahydrofuran adduct, zirconium(iv) acetylacetonate, zirconium(iv) tetrapropoxide, zirconium(iv) tetrabutoxide, zirconium 3-methyl-3-pentoxide, tetrakis(2-methyl-3-butene-2-oxy)zirconium, tetrakis(1-methoxy-2-methyl-2-propoxy) zirconium, or mixtures thereof.

Preferably, the Zn compound selected from zinc neodecanoate, zinc octoate, zinc acetylacetonate, zinc oxalate, zinc acetate, zinc propionate, zinc valerate, zinc pivalate, zinc caprylate, zinc succinate, zinc bis(2-ethylhexanoate), zinc laurate, zinc myristate, zinc bis(trifluoroacetate), zinc stearate, zinc citrate, zinc gluconate, or mixtures thereof.

Preferably, the Bi compound is selected from bismuth formate, bismuth octoate, bismuth octanoate, bismuth neodecanoate, bismuth subsalicylate, bismuth neododecanoate, bismuth neooctanoate, bismuth, bismuth trineodecanoate, bismuth triacetate, bismuth tris(2-ethylhexanoate, bismuth triflate, bismuth beta naphthol, or mixtures thereof.

Preferably, the Sn compound is selected from dibutyltin oxide, dioctyltin dilaurate, dioctyltin oxide, dibutyltin diacetate, dioctyltin diacetate, tin neodecanoate, tin octoate, tin acetylacetonate, tin oxalate, tin acetate, tin propionate, tin valerate, tin pivalate, tin caprylate, tin succinate, tin bis(2-ethylhexanoate), tin laurate, dibutyltin dilaurate, tin myristate, tin bis(trifluoroacetate), tin stearate, tin citrate, tin gluconate, or mixtures thereof.

Preferably, the Al compound is selected from aluminum neodecanoate, aluminum isopropoxide, aluminum octoate, aluminum acetylacetonate, aluminum oxalate, aluminum acetate, aluminum propionate, or mixtures thereof.

Preferably, the Ti catalyst is selected from titanium(IV)isopropoxide, titanium(IV)-ethoxide, titanium(IV)-butoxide, titanium(IV)-acetylacetonate, titaniumdiisopropoxide-bis-(acetylacetonate), or mixtures thereof.

Preferably, the catalyst is a protonic acid. Preferable protonic acid is selected from HCl, sulfuric acid, phosphoric acid, p-toluenesulfonic acid, dodecylbenzene sulfonic acid, camphor sulfonic acid, methane sulfonic acid, benzoic acid, acetic acid or mixtures thereof.

More preferably, the catalyst is combination of at least one metal catalyst as listed above and at least one protonic acid as listed above.

Preferably, the catalyst in the reaction is present in an amount in the range of 0.0001 to 30 wt. % based on total weight of compounds of formula (I), more preferably the catalyst in the reaction is present in a total amount in the range of 0.01 to 10 wt. % based on total weight of compounds of formula (I), even more preferably the catalyst in the reaction is present in a total amount in the range of 0.01 to 5 wt. % based on total weight of compounds of formula (I), most preferably the catalyst in the reaction is present in a total amount in the range of 0.01 to 3.0 wt. % based on total weight of compounds of formula (I), and in particular the catalyst in the reaction is present in a total amount in the range of 0.01 to 1 wt. % based on total weight of compounds of formula (I).

In another preferred embodiment, wherein the catalyst in the reaction is present in an amount in the range of 0.0001 to 2.0 mole equivalent based on compounds of formula (I), more preferably the catalyst in the reaction is present in a total amount in the range of 0.0001 to 1.0 mole equivalent based on compounds of formula (I), even more preferably the catalyst in the reaction is present in a total amount in the range of 0.001 to 1.0 mole equivalent based on compounds of formula (I), most preferably the catalyst in the reaction is present in a total amount in the range of 0.001 to 0.5 mole equivalent based on compounds of formula (I), and in particular the catalyst in the reaction is present in a total amount in the range of 0.001 to 0.1 mole equivalent based on compounds of formula (I).

The product of the present invention is obtained by a process carried out in presence of a solvent or in the absence of a solvent. The process may be performed in presence of a solvent or alternatively at high temperatures to ensure molten reaction conditions. When present, the solvent may be in a minimum quantity as would be necessary to ensure appropriate mixing. In context of the present invention, some light stabilizers are known to be obtainable commercially in the form of a suspension/solution, the solvent already present in such cases is sufficient and no added solvent is necessary. Preferably, the reaction mixture, in the process for obtaining the claimed product, is essentially free from any added liquid reactants or additives. Liquid additives comprise solvents, diluents and the like.

When the process is carried out in the presence of a solvent, the solvent is preferably selected from ethers, sulfones, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, more preferably the solvent is selected from ethers, ketones, water, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, even more preferably the solvent is selected from ethers, N, N-dimethylformamide, N-methyl-pyrrolidone, N-ethyl-pyrrolidone, aromatic hydrocarbons, dichloroethane, halogenated aromatic hydrocarbon, or a combination of two or more thereof, most preferably the solvent is selected from ethers, N, N-dimethylformamide, N-methyl-pyrrolidone, N-ethyl-pyrrolidone, aromatic hydrocarbons, halogenated aromatic hydrocarbon, or a combination of two or more thereof, and in particular the solvent is selected from ketones, ethers, aromatic hydrocarbons, halogenated aromatic hydrocarbon, water or a combination thereof

Preferably, the reaction is carried out in presence of a solvent in an amount in the range of 0.01 to 20 times based of total amount of formula (A) or (B), more preferably the reaction is carried out in presence of a solvent in an amount in the range of 0.1 to 10 times based of total amount of formula (I), even more preferably the reaction is carried out in presence of a solvent in an amount in the range of 0.2 to 5.0 times based of total amount of formula (I), most preferably the reaction is carried out in presence of a solvent in an amount in the range of 0.5 to 3.0 times based of total amount of formula (I), and in particular the reaction is carried out in presence of a solvent in an amount in the range of 0.5 to 2.0 times based of total amount of formula (I).

Preferably, the pH of the reaction is maintained ≤9.0, more preferably the pH of the reaction is maintained in the range of ≥0.0 to ≤8.0, even more preferably the pH of the reaction is maintained in the range of ≥4.0 to ≤8.0.

Preferably, product of the present invention is obtained by a process carried out at a temperature ≥20° C., more preferably ≥40° C., even more preferably ≥60° C., more preferably ≥80° C. Preferably, reacting at least one compound of formula (I) with at least one polyether polyol is carried out at a temperature in the range ≥20 to ≤200° C., more preferably ≥40 to ≤160° C., even more preferably ≥60 to ≤120° C. Said process may be conducted for up to 48, 24, 18, 14, 12, 10, 8, 6, 4, 3, 2, 1, or 0.5 hours, more preferably for 1 min to 12 hours, even more preferably from 30 min to 3 hours. The process may preferably be carried out in the presence of mechanical agitation such as stirring. The same may be performed by any known method using external mechanical shakers or by using magnetic stirring beads among others.

Preferably, the product obtained according to presently claimed invention has weight average molecular weight in the range of 200 to 20000, preferably in the range of 400 to 10000, more preferred in the range of 500 to 8000, even more preferred in the range of 500 to 5000 as determined by GPC using polystyrene as internal standard.

Preferably, the product obtained according to presently claimed invention has solubility greater than 10 g per 100 mL xylene at room temperature, more preferably the product obtained according to presently claimed invention has solubility greater than 20 g per 100 mL xylene at room temperature, most preferably the product obtained according to presently claimed invention has solubility greater than 30 g per 100 mL xylene at room temperature, and in particular preferably the product obtained according to presently claimed invention has solubility greater than 40 g per 100 mL xylene at room temperature.

The presently claimed invention is also directed to process for obtaining the product, or a salt thereof described hereinabove, said process comprising the step of
i) reacting the compound of formula I;
ii) with the polyether polyol,
   in the presence of at least one catalyst.

The presently claimed invention is also directed to a use of the product, or a salt described hereinabove as ultraviolet stabilizer. The use of the light stabilizer as ultraviolet stabilizer may be in personal and home care such as in cosmetics, plastics or surface coatings (such as automotive coatings).

The presently claimed invention is also directed to a composition comprising a product or a salt thereof obtained, described hereinabove.

Preferably the composition may be solvent based or water based. Typical examples of organic solvents are aliphatic, aromatic or cycloaliphatic hydrocarbons, alcohols, glycols, esters, acetates and ketones.

The presently claimed invention is also directed to a method of protecting a material or coating from light, wherein the method comprises a step of providing the product or salt thereof obtained according to presently claimed invention as UV stabilizer. Said product or salt thereof is noted to provide long term stability comparable with commonly used industry standard UV absorber (for e.g., hydroxyphenyl benzotriazole class absorber).

Preferably, the composition may be solvent based or water based. Typical examples of organic solvents are aliphatic, aromatic or cycloaliphatic hydrocarbons, alcohols, glycols, esters, acetates and ketones.

Preferably, the coatings are surface coatings such as those employed in. Preferably, the composition is an automotive coating composition.

The coating composition is preferably a laquer, in particular a stoving laquer which is used for coating automobiles (automobile finishing lacquers), for example stoving lacquers comprising alkyd/melamine resins and alkyd/acrylic/melamine resins (see H. Wagner and H. F. Sarx, “Lack-kunstharze” (1977), pages 99-123), epoxy/carboxy resins, isocyanate crosslinked acrylic polyols or polyester polyols. Other crosslinking agents include glycoluril resinsor blocked isocyanates.

The coating composition preferably contains 0.01-10 parts by weight, especially 0.05-10 parts by weight, more especially 0.1-5 parts by weight, of the product, or a salt thereof according to the invention per 100 parts by weight of a solid binder. The binders may in principle be any binders that are customary in the art, for example those described in Ullmann's Encyclopedia of Industrial Chemistry, 5th ed., Vol. A18, pp. 368-426, VCH, Weinheim 1991. The binder will generally be a film-forming binder, based on a thermoplastic or thermosetting resin, predominantly on a thermosetting resin. Examples thereof are alkyd, acrylic, polyester, phenolic, melamine, epoxy and polyurethane resins and mixtures thereof.

It may be a cold-curable or a hot-curable binder, and the addition of a curing catalyst may be advantageous. Suitable catalysts, which accelerate full curing of the binder, are described, for example, in Ullmann's Encyclopedia of Industrial Chemistry, Vol. A18, p. 469, VCH Verlagsgesellschaft, Weinheim 1991.

Multilayer systems are possible here as well, it being possible for the concentration of the stabilizers in the top layer to be higher, for example from 1 to 15 parts by weight, especially from 3 to 10 parts by weight, based on 100 parts by weight of solid binder.

### Embodiments

In the following, there is provided a list of embodiments to further illustrate the present disclosure without intending to limit the disclosure to the specific embodiments listed below.

1. A product, or a salt thereof obtainable by a process comprising the step of:
a) reacting at least one compound of formula (I) in the presence of at least one catalyst,

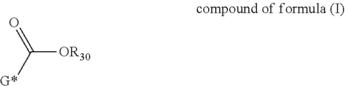

wherein G* is selected from a substituted 2-(2-hydroxyphenyl)-2H-benzotriazole group or substituted 2-hydroxyphenyl-s-triazine,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, substituted or unsubstituted, linear or branched C₁-C₂₄ heteroalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, or substituted or unsubstituted C₇-C₂₄ arylalkyl;
b) with polyether polyol obtainable by reacting a composition of:
b1) 5 to 90 wt % based on the total amount of the polyether polyol, of at least one sugar selected from C₅-C₆ sugar alcohols, monosaccharides, oligosaccharides, polysaccharides, or mixtures thereof;
b2) 5 to 90 wt % based on the total amount of the polyether polyol, of at least one alkylene oxide;
b3) 0 to 90 wt % based on the total amount of the polyether polyol, of at least one polyhydric alcohols having 2 to 4 hydroxy groups; and
b4) 0 to 15 wt % based on the total amount of the polyether polyol, of at least one optional reactant selected from fatty acid, fatty acid monoesters or mixtures thereof. 2. The product, or a salt thereof according to embodiment 2, wherein the substituted 2-hydroxyphenyl-s-triazine is selected from formula (A),

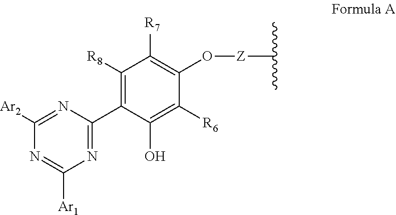

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

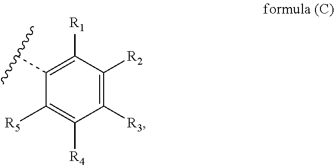

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, substituted or unsubstituted —OC₁-C₂₄ alkyl; or a moiety of formula (M),

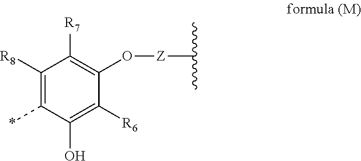

wherein the dotted line is a single bond between formula (M) and the triazinyl ring of formula (A), and Z is attached to —C(═O)OR₃₀,
R₆, R₇ and R₃ are independently of each other selected from hydrogen, or substituted or unsubstituted, linear or branched C₁-C₂₄ alkyl.

3. The product, or a salt thereof according to any of the embodiments 1 to 2, wherein substituted 2-(2-hydroxyphenyl)-2H-benzotriazole is selected from formula (B),

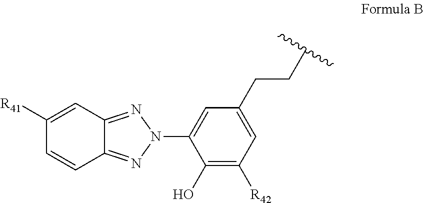

wherein R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

4. The product, or a salt thereof according to any of the embodiments 1 to 3, wherein R₃₀ is selected from hydrogen, substituted or unsubstituted, linear or branched C₁-C₂₄ alkyl, substituted or unsubstituted, linear or branched C₁-C₂₄ heteroalkyl, or substituted or unsubstituted C₇-C₂₄ arylalkyl.

5. The product, or a salt thereof according to any one of the embodiments 1 to 4, wherein Z is selected from substituted or unsubstituted, linear or branched C₁-C₈ alkylene; R₁, R₂, R₃, R₄ and R₅ are independently of each other selected from hydrogen, unsubstituted C₁-C₆ alkyl, unsubstituted C₆-C₁₂ aryl, —OH, or —OC₁-C₆ alkyl; and R₆, R₇ and R₃ are hydrogen or unsubstituted linear C₁-C₆ alkyl.

6. The product, or a salt thereof according to any one of the embodiments 1 to 5, wherein R₄₁, and R₄₂ independently of each other, are selected from hydrogen, halogen, substituted or unsubstituted, linear or branched C₁-C₆ alkyl, or substituted or unsubstituted C₇-C₂₄ arylalkyl.

7. The product, or a salt thereof according to any one of the embodiments 1 to 6, wherein the compound of formula (I) is selected from methyl 3-[3-(benzotriazol-2-yl)-5-tert-butyl-4-hydroxyphenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxyphenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, octyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate, 6-methylheptyl 2-[4-[4,6-bis(4-phenylphenyl)-1,3,5-triazin-2-yl]-3-hydroxy-phenoxy]propanoate, octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, methyl 2-[4-[4,6-bis(2,4-dimethylphenyl)-1,3,5-triazin-2-yl]-2-methyl-3-hydroxy-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof.

8. The product, or a salt thereof according to embodiment 7, wherein the compound of formula (I) is selected from methyl 2-[4-[4,6-bis(2,4-dimethylphenyl)-1,3,5-triazin-2-yl]-3-hydroxyphenoxy]propanoate, methyl 2-[4-(4,6-diphenyl-1,3,5-triazin-2-yl)-3-hydroxy-phenoxy]propanoate, octyl 2-[4-(4,6-diphenyl-1,3,5-triazin-2-yl)-3-hydroxy-phenoxy]propanoate, methyl 2-[4-[4,6-bis(4-phenylphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate, 6-methylheptyl 2-[4-[4,6-bis(4-phenylphenyl)-1,3,5-triazin-2-yl]-3-hydroxy-phenoxy]propanoate, methyl 2-[4-[4,6-bis(2,4-dimethylphenyl)-1,3,5-triazin-2-yl]-2-methyl-3-hydroxy-phenoxy]propanoate, methyl 3-[3-(benzotriazol-2-yl)-5-tert-butyl-4-hydroxyphenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

9. The product, or a salt thereof according to any one of the embodiments 1 to 8, wherein the weight ratio of compound of formula (I) to the polyether polyol is in the range of 1 to 10 to 10 to 1.

10. The product, or a salt thereof according to any one of the embodiments 1 to 9, wherein the at least one catalyst is selected from protonic acids, Sn compound, Zr compound, Bi compound, Zn compound, Al compound, Ti compound, or a combination of two or more thereof.

11. The product, or a salt thereof according to any one of the embodiments 1 to 10, wherein the polyhydric alcohol is selected from glycerol, monopropylene glycol, dietheylene glycol, triethylene glycol, dipropylene glycol, erythritol, pentaerythritol, trimethylolpropane, or mixtures thereof.

12. The product, or a salt thereof according to any one of the embodiments 1 to 11, wherein the alkylene oxide is selected from C₂-C₂₀ alkylene oxide, preferably C₂-C₆ alkylene oxide.

13. The product, or a salt thereof according to any one of the embodiments 1 to 12, wherein the polyether polyol has viscosity in the range from 1000 to 50000 mPa·S, preferably from 2000 to 45000 mPa·S, measured at 25° C. according to DIN EN 12092.

14. The product, or a salt thereof according to any one of the embodiments 1 to 13, wherein the polyether polyol has an OH number in the range from 50 to 2000 mg KOH/g, preferably from 100 to 1500 mg KOH/g, measured according to DIN 53 240(1971 December).

15. The product, or a salt thereof according to any one of the embodiments 1 to 14, wherein the polyether polyol has a weight average molecular weight from 100 to 2500 g/mol, preferably from 150 to 1000 g/mol, estimated with determined by GPC using polystyrene as internal standard.

16. The product, or a salt thereof according to any one of the embodiments 1 to 15, wherein the polyether polyol has a weight average molecular weight from 150 to 1000 g/mol, estimated with determined by GPC using polystyrene as internal standard.

17. The product, or a salt thereof according to any one of the embodiments 1 to 16 has weight average molecular weight in the range of 500 to 20000, estimated with determined by GPC using polystyrene as internal standard.

18. The product, or a salt thereof according to any one of the embodiments 1 to 17 has solubility greater than 10 g per 100 mL xylene at room temperature.

19. A process for obtaining the product, or a salt thereof according to any one of the embodiments 1 to 18, said process comprising the step of
a. reacting the compound of formula I;
b. with the polyether polyol,
in the presence of at least one catalyst.

20. Use of the product or a salt thereof according to any one of the embodiments 1 to 18 as ultraviolet stabilizer.

21. A composition comprising a product or a salt thereof according to any one of the embodiments 1 to 18.

22. A method of protecting a material or coating from light, wherein the method comprises a step of providing the product according to any one of the embodiments 1 to 18 or salt thereof as UV stabilizer.

The presently claimed invention is illustrated in detail by non-restrictive working examples which follow. More particularly, the test methods specified hereinafter are part of the general disclosure of the application and are not restricted to the specific working examples.

### EXAMPLES

Methyl 3-[3-(benzotriazol-2-yl)-5-tert-butyl-4-hydroxy-phenyl]propanoate (B02) is available from Alfa Chemistry, USA.

2-[4-[4,6-bis(2,4-dimethylphenyl)-1,3,5-triazin-2-yl]-3-hydroxyphenoxy]-propionic acid methyl ester (A01), 2-[4-[4,6-bis([1,1′-biphenyl]-4-yl)-1,3,5-triazin-2-yl]-3-hydroxyphenoxy]-propionic acid methyl ester (A02) and mixtures of A01 and A02 were produced according to a procedure as described in patent application WO2022233760 A1.

Dibutyltin dilaurate (DBTL) and para-toluenesulfonic acid monohydrate are available from Sigma-Aldrich, Germany.

Lupranol® 3405/1 (sucrose, oligomeric reaction product with propylene oxide and glycerol having OH number 450 mg KOH/g) is available from BASF, Germany.

Lupranol® 3408/1 (sucrose, oligomeric reaction product with propylene oxide and glycerol having OH number 420 mg KOH/g) is available from BASF, Germany.

Lupranol® 3409/1 (sucrose, oligomeric reaction product with propylene oxide and glycerol having OH number 430 mg KOH/g) is available from BASF, Germany.

Lupranol® 3422 (alkoxylation product of sorbitol and propylene oxide having OH number 490 mg KOH/g) is available from BASF, Germany.

Lupranol® 3423 (sucrose, oligomeric reaction product with propylene oxide and glycerol having OH number 490 mg KOH/g) is available from BASF, Germany.

Lupranol® 3424 (sucrose, oligomeric reaction products with pentaerythritol, propylene oxide and diethylene glycol having OH number 403 mg KOH/g) is available from BASF, Germany.

Pernil® ME V 05 (oleic acid methyl ester) is available from BASF, Germany.

### Example 1

A 100 mL glass flask containing a magnetic stir bar was placed in an agitating heating block. Lupranol 3405/1 (14.5 grams), dibutyltin dilaurate (0.075 grams) and compound of formula B02 (32.5 grams, 0.09 mole) were transferred into the flask. Under an argon flow, the flask content was heated to 180° C. and a 20 mbar vacuum was applied. After 5.5 h stirring dibutyltin dilaurate (0.025 grams) was added. After 15.5 h stirring Lupranol 3405-1 (2.01 grams) and after 22.0 h stirring Lupranol 3405-1 (2.05 grams) were added. A HPLC analysis after 33.5 h stirring indicated a conversion of >97% of compound of formula B02. The flask contents were discharged and cooled yielding 37.3 grams of the UV absorbing polymer as a clear, brownish melt.

### Example 2

A 100 mL glass flask containing a magnetic stir bar was placed in an agitating heating block. Lupranol 3423 (12.3 grams), dibutyltin dilaurate (0.079 grams) and compound of formula B02 (34.6 grams, 0.10 mole) were transferred into the flask. Under an argon flow, the flask content was heated to 180° C. and a 20 mbar vacuum was applied. After 5.5 h stirring dibutyltin dilaurate (0.026 grams) was added. After 15.5 h stirring Lupranol 3405-1 (1.82 grams) and after 22.0 h stirring Lupranol 3405-1 (2.38 grams) were added. A HPLC analysis after 33.5 h stirring indicated a conversion of >95% of compound of formula B02. The flask contents were discharged and cooled yielding 41.3 grams of the UV absorbing polymer as a clear, orange-brownish melt.

### Example 3

A 100 mL glass flask containing a magnetic stir bar was placed in an agitating heating block. Lupranol 3423 (15.0 grams), dibutyltin dilaurate (0.097 grams) and compound of formula B02 (31.8 grams, 0.09 mole) were transferred into the flask. Under an argon flow, the flask content was heated to 180° C. and a 20 mbar vacuum was applied. After 5.5 h stirring dibutyltin dilaurate (0.027 grams) was added. After 15.5 h stirring Lupranol 3405-1 (1.27 grams) and after 22.0 h stirring Lupranol 3405-1 (0.94 grams) were added. A HPLC analysis after 33.5 h stirring indicated a conversion of 98% of compound of formula B02. The flask contents were discharged and cooled yielding 41.7 grams of the UV absorbing polymer as a clear, orange-brownish melt.

The same product was also obtained in acceptable yield when using tin(II)-ethylhexanoate as catalyst.

### Example 4

A 100 mL glass flask containing a magnetic stir bar was placed in an agitating heating block. Lupranol 3423 (14.5 grams), Pernil ME V 05 (2.0 grams), dibutyltin dilaurate (0.096 grams) and compound of formula B02 (30.7 grams, 0.09 mole) were transferred into the flask. Under an argon flow, the flask content was heated to 180° C. and a 20 mbar vacuum was applied. After 5.5 h stirring dibutyltin dilaurate (0.024 grams) was added. After 15.5 h stirring Lupranol 3405-1 (1.50 grams) and after 22.0 h stirring Lupranol 3405-1 (1.39 grams) were added. A HPLC analysis after 33.5 h stirring indicated a conversion of >97% of compound of formula B02. The flask contents were discharged and cooled yielding 43.9 grams of the UV absorbing polymer as a clear, orange-brownish melt.

TABLE 1
details of examples 1-4.

| | Absorbance A in dichloromethane | | | | | Solubility at |
|---|---|---|---|---|---|---|
| | at 20 mg/L | | | Mn (GPC) | Mw (GPC) | room temperature |
| | at 320 | at 340 | at 360 | [Da] | [Da] | [w-%] |
| Example | nm | nm | nm | Da | Da | xylene |
| Example 1 | 0.50 | 0.61 | 0.46 | 1157 | 1522 | 65 |
| Example 2 | 0.48 | 0.58 | 0.45 | 1001 | 1357 | 65 |
| Example 3 | 0.50 | 0.60 | 0.46 | 1022 | 1326 | 65 |
| Example 4 | 0.45 | 0.55 | 0.42 | 1037 | 1365 | 65 |

### Example 5

A 100 mL glass flask containing a magnetic stir bar was placed in an agitating heating block.

Lupranol 3408/1 (12.0 grams), dibutyltin dilaurate (0.063 grams) and compound of formula B02 (28.3 grams) were transferred into the flask. Under an argon flow, the flask content was heated to 180° C. and a 20 mbar vacuum was applied. After 9 h stirring dibutyltin dilaurate (0.03 grams) was added. After 12 h stirring Lupranol 3408/1 (1.83 grams), after 26 h stirring Lupranol 3408/1 (2.04 grams) and after 30 h stirring Lupranol 3408/1 (2.08 grams) was added. A HPLC analysis after 36 h stirring indicated a conversion of >98% of compound of formula B02. The flask contents were discharged and cooled yielding 41.8 grams of the UV absorbing polymer as a clear, brownish melt.

Examples 6-11 were produced in the same manner as described in Example 5. In Table 2 the amounts of compound B02 and the polyether polyol are indicated as well as the reaction time and yield of products.

TABLE 2
details for examples 5-11

| | Compound | | | Reaction | | |
|---|---|---|---|---|---|---|
| Table 2 | B-02 | Polyether Polyol | | Time | Product | |
| Examples | [g] | [g] | Lupranol | [h] | [g] | aspect |
| # 5 | 28.3 | 18 | 3408/1 | 36 | 41.8 | clear, brownish |
| | | | | | | melt |
| # 6 | 25.4 | 18.5 | 3409/1 | 30 | 35.1 | clear, brownish |
| | | | | | | melt |
| # 7 | 28.3 | 19.2 | 3409/1 | 36 | 42.9 | clear, brownish |
| | | | | | | melt |
| # 8 | 28.3 | 16.9 | 3422 | 15 | 39.7 | clear, light |
| | | | | | | orange melt |
| # 9 | 30.2 | 20 | 3422 | 20 | 46.2 | clear, light |
| | | | | | | orange melt |
| # 10 | 29.7 | 14.6 | 3423 | 15 | 34.1 | clear, brownish |
| | | | | | | melt |
| # 11 | 21.2 | 12 | 3424 | 15 | 29.5 | clear, light |
| | | | | | | orange melt |

Example 11 was also obtained in acceptable yield when using zirconium neodecanoate (TIE KAT 818, TIE Chemicals, Germany), aluminum triisopropylate or bismuth neodecanoate (TIE KAT 716, TIE Chemicals, Germany) as catalyst replacing dibutyltin dilaurate.

In Table 3 the absorbance, GPC and solubility data are listed.

TABLE 3

| | Absorbance A in dichloromethane | | | | | Solubility at |
|---|---|---|---|---|---|---|
| | at 20 mg/L | | | Mn | Mw* | room temperature |
| | at 320 | at 340 | at 360 | (GPC) | (GPC) | g in 100 mL |
| Examples | nm | nm | nm | Da | Da | xylene |
| # 5 | 0.44 | 0.53 | 0.41 | 1172 | 1489 | 85 |
| # 6 | 0.42 | 0.51 | 0.39 | 1314 | 1663 | 85 |
| # 7 | 0.43 | 0.53 | 0.40 | 1351 | 1694 | 85 |
| # 8 | 0.46 | 0.56 | 0.43 | 1174 | 1404 | 85 |
| # 9 | 0.45 | 0.54 | 0.42 | 1142 | 1343 | 85 |
| # 10 | 0.49 | 0.59 | 0.46 | 1054 | 1393 | 85 |
| # 11 | 0.49 | 0.59 | 0.46 | 995 | 1082 | 85 |

*Mw GPC—weight average molecular weight of product as estimated by GPC with styrene as standard.

### Example 12

A 100 mL glass flask containing a magnetic stir bar was placed in an agitating heating block. Lupranol 3408/1 (4.6 grams), dibutyltin dilaurate (0.075 grams) and compound of formula A01 (2.0 grams) were transferred into the flask. Under an argon flow, the flask content was heated to 200° C. and a 20 mbar vacuum was applied. Additional amounts of compound of formula A01 were added after 4 h stirring (2.0 grams) and after 8 h stirring (2.0 grams). A HPLC analysis after 15 h stirring indicated a conversion of 96.6% of compound of formula A01. The flask contents were discharged and cooled yielding 8.4 grams of the UV absorbing polymer as a clear, brownish melt.

### Example 13

A 100 mL glass flask containing a magnetic stir bar was placed in an agitating heating block. Lupranol 3409/1 (8.6 grams), dibutyltin dilaurate (0.1 grams) and compound of formula A02 (2.0 grams) were transferred into the flask. Under an argon flow, the flask content was heated to 200° C. and a 20 mbar vacuum was applied. Additional amounts of compound of formula A02 were added after 4 h stirring (2.0 grams) and after 8 h stirring (2.0 grams). After 21 h stirring the flask contents were discharged and cooled yielding 12.5 grams of the UV absorbing polymer as a clear, brownish melt.

### Example 14

A 100 mL glass flask containing a magnetic stir bar was placed in an agitating heating block. Lupranol 3422 (6.2 grams), dibutyltin dilaurate (0.1 grams) and compound of formula A02 (2.0 grams) were transferred into the flask. Under an argon flow, the flask content was heated to 200° C. and a 20 mbar vacuum was applied. Additional amounts of compound of formula A02 were added after 4 h stirring (2.0 grams) and after 8 h stirring (2.0 grams). After 21 h stirring the flask contents were discharged and cooled yielding 10.2 grams of the UV absorbing polymer as a clear, orange melt.

### Example 15

A 100 mL glass flask containing a magnetic stir bar was placed in an agitating heating block. Lupranol 3422 (3.6 grams), dibutyltin dilaurate (0.2 grams) and a mixture (4 grams) containing compound of formula A01 (44%) and compound of formula A02 (54%) were transferred into the flask. Under an argon flow, the flask content was heated to 200° C. and a 20 mbar vacuum was applied. Additional amounts of Lupranol 3422 were added after 10 h stirring (2.1 grams) and after 15 h stirring (3.7 grams). After 21 h stirring the flask contents were discharged and cooled yielding 12.3 grams of the UV absorbing polymer as a clear, brownish melt.

### Example 16

A 100 mL glass flask containing a magnetic stir bar was placed in an agitating heating block. Lupranol 3423 (3.2 grams), dibutyltin dilaurate (0.2 grams) and a mixture (4 grams) containing compound of formula A01 (44%) and compound of formula A02 (54%) were transferred into the flask. Under an argon flow, the flask content was heated to 200° C. and a 20 mbar vacuum was applied. Additional amounts of Lupranol 3422 were added after 10 h stirring (1.34 grams) and after 15 h stirring (1.7 grams). After 21 h stirring the flask contents were discharged and cooled yielding 9.4 grams of the UV absorbing polymer as a clear, brownish melt.

### Example 17

A 100 mL glass flask containing a magnetic stir bar was placed in an agitating heating block. Lupranol 3424 (5.1 grams), dibutyltin dilaurate (0.2 grams) and a mixture (4 grams) containing compound of formula A01 (44%) and compound of formula A02 (54%) were transferred into the flask. Under an argon flow, the flask content was heated to 200° C. and a 20 mbar vacuum was applied. Additional amounts of Lupranol 3424 were added after 10 h stirring (0.9 grams) and after 15 h stirring (3.6 grams). After 21 h stirring the flask contents were discharged and cooled yielding 21.1 grams of the UV absorbing polymer as a clear, brownish melt.

### Example 18

A 100 mL glass flask containing a magnetic stir bar was placed in an agitating heating block. Compound of formula B01 (7.6 grams), Lupranol 3424 (2.20 grams), p-toluenesulfonic acid monohydrate (0.08 grams) and toluene (20 grams) were transferred into the flask. Under an argon flow, the flask content was heated under reflux. Additional amounts of Lupranol 3424 were added after 4 h stirring (0.5 grams) and after 5 h stirring (1.0 grams). A HPLC analysis after 7 h stirring indicated a conversion of 97% of compound of formula B01. The flask contents were discharged and cooled yielding 8.0 grams of the UV absorbing polymer as a viscous, yellow liquid.

In Table 4 the absorbance, GPC and solubility data of examples 12-18 are summarized.

TABLE 4

| | | | | | | Solubility at |
|---|---|---|---|---|---|---|
| | Absorbance A in dichloromethane | | | Mn | Mw* | room temperature |
| | at 20 mg/L | | | (GPC) | (GPC) | g in 100 mL |
| Examples | at 320 nm | at 340 nm | at 360 nm | Da | Da | xylene |
| # 12 | 0.57 | 0.47 | 0.14 | 1109 | 1532 | 85% |
| # 13 | 0.88 | 0.69 | 0.21 | 1048 | 1387 | 85% |
| # 14 | 1.14 | 0.88 | 0.27 | 948 | 1211 | 85% |
| # 15 | 0.46 | 0.40 | 0.12 | 706 | 921 | 85% |
| # 16 | 0.61 | 0.52 | 0.16 | 781 | 1094 | 85% |
| # 17 | 0.46 | 0.40 | 0.12 | 628 | 795 | 85% |
| # 18 | 0.50 | 0.62 | 0.47 | 1008 | 1145 | 85% |

*Mw GPC—weight average molecular weight of product as estimated by GPC with styrene as standard.

As can be observed from Tables 1, 3 and 4, the product obtained reveal significantly high xylene solubility, while incorporation of biologically relevant sugars improves the environmental impact of the UV absorbers.

### Application Tests:

The products of the present invention are useful as UV stabilizers and were tested (examples 1-4 as described above) in a thermo-setting acrylic clear coating having the following composition:

| | |
|---|---|
| Viacryl ® SC 303/65 XB¹⁾ | 30.14 |
| Viacryl ® SC 370/75SNA²⁾ | 25.58 |
| Maprenal ® MF 650³⁾ | 29.90 |
| Butyl acetate/butanol (37/8) | 4.74 |
| Isobutanol | 5.34 |
| Solvesso ® 150⁴⁾ | 2.98 |
| Baysilon ® MA⁵⁾ | 1.31 |
| | 100.00 g |

¹⁾acrylate resin (60% solution in xylene/butanol 26:9); Allnex
²⁾acrylate resin (75% solution in Solvesso 150⁴⁾); Allnex
³⁾melamine resin (55% solution in isobutanol); Ineos melamines
⁴⁾aromatic hydrocarbon mixture, boiling range 182-203° C.; ExxonMobil Chemical
⁵⁾1% in Solvesso 150⁴⁾; Borchers
The solid content of the composition is 53%.

The product of the present invention (UV stabilizer) to be tested (examples 1-4 as described above) is added to the clear coating in the quantity shown in table 5, based on the solid content of the coating. Different dosage levels are needed, as the UV absorbing chromophore content in the tested compounds are different and needed to be adjusted for evaluation of the stabilization effect. The coating formulations are additionally admixed with 1% by weight, based on the solid content of the coating, of a co-stabilizer (compound z) of the formula (Tinuvin 123)

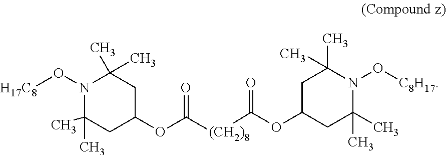

The clear coating is applied on a white coil-coated panel resulting after cure (130° C. for 30 minutes) in a dry film thickness of around 40 μm.

The coated panels are subjected to artificial weathering cycles according to SAE-J2527 in Xe-WOM weathering device from Atlas Corp. After certain exposure time the gloss at 20° is measured and then the exposure is continued. The retained gloss is an indication of the UV stabilization effect of the tested UV absorbers in the coating film, the lower gloss the more surface degradation takes place.

TABLE 5
Accelerated weathering results: gloss
(20°) after x exposure time (hours)

| | | Exposure time (hours) | | | | | | |
|---|---|---|---|---|---|---|---|---|
| | | 0 | 1000 | 2000 | 3000 | 4000 | 5000 | 6000 |
| UV stabilizer^{#} | | h | h | h | h | h | h | h |
| Without UV | — | 82 | 80 | 74 | 63 | nm* | nm* | nm* |
| stabilizer | | | | | | | | |
| Tinuvin ® 1130 | 2.0 | 82 | 76 | 73 | 67 | 63 | 58 | 51 |
| Tinuvin ® 384-2 | 1.4 | 81 | 79 | 75 | 70 | 68 | 61 | 56 |
| Example 1 | 1.5 | 84 | 78 | 74 | 68 | 65 | 59 | 54 |
| Example 2 | 1.7 | 82 | 75 | 70 | 66 | 62 | 56 | 51 |
| Example 3 | 1.5 | 84 | 80 | 76 | 73 | 70 | 64 | 54 |
| Example 4 | 1.7 | 84 | 80 | 74 | 69 | 64 | 60 | 56 |

*not measured due to strong crack formation;
^{#}amounts in wt.-% with respect to solid content.

The blank samples (without any stabilizer) revealed strong degradation of the coating film due to insufficient stabilization. The UV stabilizing property indicated by the gloss of the product of presently claimed invention was comparable to the standard Tinuvin® 1130 or Tinuvin® 384-2.

1.-17. (canceled)

18. A product, or a salt thereof obtained by a process comprising the step of:
a) reacting at least one compound of formula (I) in the presence of at least one catalyst, 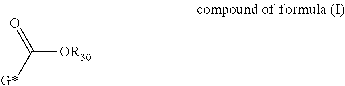
wherein G* is selected from a substituted 2-(2-hydroxyphenyl)-2H-benzotriazole group or substituted 2-hydroxyphenyl-s-triazine,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, substituted or unsubstituted, linear or branched C₁-C₂₄ heteroalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, or substituted or unsubstituted C₇-C₂₄ arylalkyl;
b) with polyether polyol obtained by reacting a composition of:
b1) 5 to 90 wt % based on the total amount of the polyether polyol, of at least one sugar selected from C₅-C₆ sugar alcohols, monosaccharides, oligosaccharides, polysaccharides, or mixtures thereof;
b2) 5 to 90 wt % based on the total amount of the polyether polyol, of at least one alkylene oxide;
b3) 0 to 90 wt % based on the total amount of the polyether polyol, of at least one polyhydric alcohols having 2 to 4 hydroxy groups; and
b4) 0 to 15 wt % based on the total amount of the polyether polyol, of at least one optional reactant selected from fatty acid, fatty acid monoesters or mixtures thereof.

19. The product, or a salt thereof according to claim 18, wherein the substituted 2-hydroxyphenyl-s-triazine is selected from formula (A), 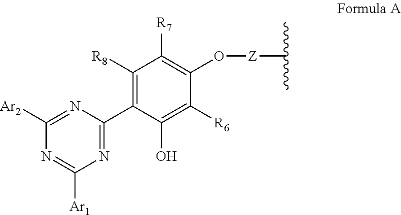
wherein Z is selected from substituted or unsubstituted, linear or branched C1-C30 alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C), 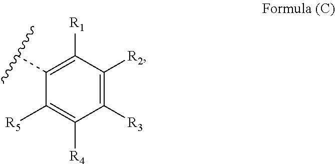
wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, substituted or unsubstituted —OC₁-C₂₄ alkyl; or a moiety of formula (M), 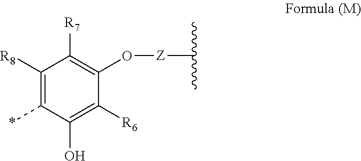
wherein the dotted line is a single bond between formula (M) and the triazinyl ring of formula (A), and Z is attached to —C(═O)OR₃₀,
R₆, R₇ and R₈ are independently of each other selected from hydrogen, or substituted or unsubstituted, linear or branched C₁-C₂₄ alkyl.

20. The product, or a salt thereof according to claim 18, wherein substituted 2-(2-hydroxyphenyl)-2H-benzotriazole is selected from formula (B), 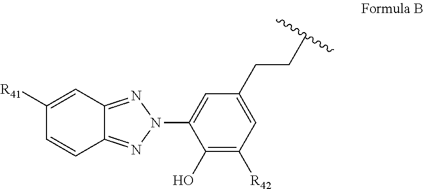
wherein R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

21. The product, or a salt thereof according to claim 18, wherein Z is selected from substituted or unsubstituted, linear or branched C₁-C₈ alkylene; R₁, R₂, R₃, R₄ and R₅ are independently of each other selected from hydrogen, unsubstituted C₁-C₆ alkyl, unsubstituted C₆-C₁₂ aryl, —OH, or —OC₁-C₆ alkyl; and R₆, R₇ and R₈ are hydrogen.

22. The product, or a salt thereof according to claim 18, wherein R₄₁, and R₄₂ independently of each other, are selected from hydrogen, halogen, substituted or unsubstituted, linear or branched C₁-C₆ alkyl.

23. The product, or a salt thereof according to claim 18, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxyphenyl]propanoate, methyl 2-[4-[4,6-bis(2,4-dimethylphenyl)-1,3,5-triazin-2-yl]-3-hydroxyphenoxy]propanoate, methyl 2-[4-[4,6-bis(4-phenylphenyl)-1,3,5-triazin-2-yl]-3-hydroxyphenoxy]propanoate, methyl 2-[4-(4,6-diphenyl-1,3,5-triazin-2-yl)-3-hydroxyphenoxy]propanoate, octyl 2-[4-(4,6-diphenyl-1,3,5-triazin-2-yl)-3-hydroxyphenoxy]propanoate, ethyl 2-[4-[4,6-bis(2,4-dimethylphenyl)-1,3,5-triazin-2-yl]-3-hydroxyphenoxy]-3-butoxy-propanoate, ethyl 2-[4-(4,6-diphenyl-1,3,5-triazin-2-yl)-3-hydroxyphenoxy]octanoate, methyl 2-[4-(4,6-diphenyl-1,3,5-triazin-2-yl)-3-hydroxyphenoxy]butanoate, methyl 2-[4-[4,6-bis(2,4-dimethylphenyl)-1,3,5-triazin-2-yl]-3-hydroxyphenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, octyl 2-[4-[4,6-bis[2-hydroxy-4-(1-methyl-2-octoxy-2-oxoethoxy)phenyl]-1,3,5-triazin-2-yl]-3-hydroxy-phenoxy]propanoate, 6-methylheptyl 2-[4-[4,6-bis(4-phenylphenyl)-1,3,5-triazin-2-yl]-3-hydroxy-phenoxy]propanoate, octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, methyl 2-[4-[4,6-bis(2,4-dimethylphenyl)-1,3,5-triazin-2-yl]-2-methyl-3-hydroxy-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxyphenoxy]butanoate, methyl 2-[4-[4,6-bis(2,4-dimethylphenyl)-1,3,5-triazin-2-yl]-3-hydroxyphenoxy]butanoate, ethyl 2-[4-[4,6-bis(4-phenylphenyl)-1,3,5-triazin-2-yl]-3-hydroxyphenoxy]propanoate, methyl 2-[4-[4,6-bis[2-hydroxy-4-(2-methoxy-1-methyl-2-oxoethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof.

24. The product, or a salt thereof according to claim 18, wherein the weight ratio of compound of formula (I) to the polyether polyol is in the range of 1 to 10 to 10 to 1.

25. The product, or a salt thereof according to claim 18, wherein the catalyst is selected from protonic acids, Sn compound, Zr compound, Bi compound, Zn compound, Al compound, Ti compound, or a combination of two or more thereof.

26. The product, or a salt thereof according to claim 18, wherein the polyhydric alcohol is selected from glycerol, monopropylene glycol, dietheylene glycol, triethylene glycol, dipropylene glycol, erythritol, pentaerythritol, trimethylolpropane, or mixtures thereof.

27. The product, or a salt thereof according to claim 18, wherein the alkylene oxide is selected from C₂-C₂₀ alkylene oxide.

28. The product, or a salt thereof according to claim 18, wherein the polyether polyol has an OH number in the range from 50 to 2000 mg KOH/g.

29. The product, or a salt thereof according to claim 18, wherein the polyether polyol has a weight average molecular weight from 100 to 2500 g/mol estimated with determined by GPC using polystyrene as internal standard.

30. The product, or a salt thereof according to claim 18 has weight average molecular weight in the range of 500 to 20000, estimated with determined by GPC using polystyrene as internal standard.

31. A process for obtaining the product, or a salt thereof according to claim 18, said process comprising the step of
a. reacting the compound of formula I;
b. with the polyether polyol,
in the presence of at least one catalyst.

32. An ultraviolet stabilizer comprising the product or salt according to claim 18.

33. A composition comprising a product or a salt thereof according to claim 18.

34. A method of protecting a material or coating from light, wherein the method comprises a step of providing the product according to claim 18 or salt thereof as UV stabilizer.
